(19) United States
(12) Patent Application Publication
(10) Pub. No.: US 2024/0277214 A1
(43) Pub. Date: Aug. 22, 2024

(54) WIRELESS CAMERA ASSEMBLY

(71) Applicant: Integrated Endoscopy, Inc., Irvine, CA (US)

(72) Inventor: Bradley Jay Sharp, Irvine, CA (US)

(21) Appl. No.: 18/445,000

(22) Filed: Feb. 19, 2024

Related U.S. Application Data
(60) Provisional application No. 63/486,100, filed on Feb. 21, 2023.

(51) Int. Cl. A61B 1/00; A61B 1/06; H04N 23/50; H04N 23/51; H04N 23/57

(52) U.S. Cl. CPC A61B 1/00142; A61B 1/00016; A61B 1/00032; A61B 1/00096; A61B 1/00103; A61B 1/00124; A61B 1/00128; A61B 1/00131; A61B 1/0684; H04N 23/51; H04N 23/555; H04N 23/57

(57) ABSTRACT

A wireless camera assembly includes a disposable cover that removably receives and encloses a wireless camera (e.g., wireless camera module). The cover has a connector that removably couples to an endoscope. The cover can be disposed after use (e.g., after a single use or procedure), allowing the wireless camera (e.g., wireless camera module) to be reused in multiple surgical procedures and to not require autoclaving (or other sterilization) between surgical procedures.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND

### Field

The present disclosure is directed to wireless cameras for use in surgical procedures, such as for use with an endoscope in an endoscopic procedure, and more particularly to a wireless camera assembly including a disposable cover for the wireless camera.

### Description of the Related Art

Equipment used in surgical procedures has to be sterilized between uses, such as via an autoclave. However, autoclaving can be hard on equipment, reducing their life of operation.

### SUMMARY

In accordance with one aspect of the disclosure, a disposable sterile cover for a wireless camera is provided that can surround the camera during use and can be disposed after use (e.g., after a single use or procedure), allowing the camera to not require autoclaving (or other sterilization) between surgical procedures. The camera can couple to an endoscope (e.g., to an eyecup of an endoscope). Optionally, the cover is of resilient material. Optionally, the cover has a clamshell form.

In accordance with another aspect of the disclosure, a disposable cover for a wireless camera module is provided that can receive and enclose the camera module to provide a camera. The cover can include batteries can removably couple to an endoscope. The cover can be disposed after use (e.g., after a single use or procedure), with or without the batteries, allowing the camera module (and/or batteries) to be reused and to not require autoclaving (or other sterilization) between surgical procedures.

In some aspects, the techniques described herein relate to a wireless camera assembly, including: a disposable cover including a housing and a hatch movable relative to the housing between an open position allowing access to a cavity in the housing and closed position to close the housing, the housing including a connector configured to couple to a proximal end of an endoscope, the housing having a window or opening via which an image passes into the housing from the endoscope, and the disposable cover configured to receive a wireless camera in the cavity of the disposable cover when the hatch is in the open position, the cover configured to align an imaging device of the wireless camera with the window of the housing and with the endoscope when the endoscope is coupled to the connector and when the wireless camera is inserted into the cavity.

In some aspects, the techniques described herein relate to a wireless camera assembly, including: a disposable cover including a housing and a hatch movable relative to the housing between an open position allowing access to a cavity in the housing and closed position to close the housing, the housing including a connector; an endoscope having a proximal end coupled to the connector of the housing, the endoscope being manually rotatable by a user about an axis of an endoscope tube of the endoscope with a same hand holding the cover to vary a view angle of the endoscope; and a wireless camera removably insertable in the cavity of the disposable cover, the camera insertable into the housing or removable from the housing when the hatch is in the open position, an imaging device of the wireless camera configured to align with the endoscope when the wireless camera is inserted into the cavity, the imaging device configured to receive an image from the endoscope.

### BRIEF DESCRIPTION OF THE DRAWINGS

FIG. 1 is a schematic side view of an endoscope.

FIG. 2 is a schematic cross-sectional view of a cover for a wireless camera coupleable to an eye cup of an endoscope.

FIG. 3 is a schematic cross-sectional view of a cover for a wireless camera for an endoscope.

FIG. 4 is a schematic view of a cover that removably receives a camera module and is coupleable to an endoscope.

FIG. 4A shows a schematic partial view of an indexing slot of the camera cover that removably receives a camera module.

FIG. 4B shows a schematic partial view of an indexing slot of the camera cover with the camera module installed in the cover.

FIG. 4C show a schematic view of the camera module.

FIG. 5 is a schematic view of a cover that removably receives a camera module and is coupleable to an endoscope.

FIG. 6 is a schematic view of the cover in a closed position enclosing the camera module and coupled to an endoscope.

FIG. 6A is a schematic partial view of a portion of the cover that couples to an endoscope.

FIG. 7 is a schematic view of the cover in a closed position enclosing the camera module and coupled to an endoscope.

### DETAILED DESCRIPTION

FIG. 1 illustrates an endoscope 100 such as, for example, an arthroscope, which can be used to inspect one or more portions of a human body. For example, in one implementation, the endoscope 100 can be employed to inspect the interior of a knee (e.g., during an arthroscopic procedure on the knee).

The endoscope 100 (e.g., arthroscope) can have a housing 10 (e.g., defined by a front housing portion 10A and a rear housing portion 10B), an endoscope tube or elongate member 20 (e.g., a rigid endoscope tube), and an eyepiece (e.g., eyecup, eye shield) 30. The handle 15 of the endoscope 100 (e.g., defined by the housing 10, such as by the front housing portion 10A and the rear housing portion 10B) can extend orthogonal (e.g., perpendicular to the axis of the endoscope tube 20). The endoscope tube or elongate member 20 can optionally be made of stainless steel. The front housing portion 10A can include a connector 50 (e.g., at its distal end). The connector 50 can couple with a corresponding connector in a cannula (not shown) that slides over and releasably couples with the connector 50. In one example, the endoscope 100 has a user interface 70 (e.g., button, switch) on the handle 15 (e.g., on the front housing portion 10A), which can be actuated by a user to operate (e.g., turn on, turn off) the endoscope 100 (e.g., turn on or off the light source of the endoscope). In other examples, as further discussed below, the user interface is excluded from the endoscope. In some implementations, the endoscope 100 can advantageously be disposable (e.g., single use, disposed after a single use).

The endoscope tube or elongate member 20 can have one or more (e.g., a plurality) of lenses disposed within an elongate tubular structure having proximal and distal ends. In some implementations, the lenses can be made of glass. In other implementations, the lenses can be made of plastic, such as polymethyl methacrylate (PMMA), Cyclic olefin copolymer (COC), or Cyclic olefin polymer (COP). The lenses can relay an image of features in the body located at the distal end of the endoscope 100 to the proximal end of the endoscope 100. In some implementations, the eyepiece 30 or other optics may be used to view the image. In other embodiments, disclosed further below, the eyepiece 30 is excluded from the endoscope 100. In certain implementations, the endoscope 100 may additionally have a light source that is sized, and positioned so as to be inserted into the body cavity to provide illumination therein. In some embodiments, for example, this light source is disposed at the distal end of the endoscope tube or elongate member 20. In some implementations, this light source comprises at least one solid state emitter, such as a light emitting diode (LED), located at the distal end of the endoscope. As shown in FIG. 1, the distal end of the endoscope 100 can be angled (e.g., beveled) so that rotation of the endoscope 100 about the axis of the endoscope tube 20 changes the viewing angle of the endoscope 100 (e.g., to view a different portion of the surgical site in which the endoscope is inserted). In some examples, the endoscope 100 includes circuitry (e.g., a printed circuit board or PCB) and batteries (e.g., in the handle 15 between the front housing portion 10A and the rear housing portion 10B). In another example, as further discussed below, the endoscope excludes any circuitry and/or batteries and the light source (e.g., LEDs) of the endoscope are powered by batteries in a cover of the camera.

In operation, light emitted from the light source illuminates various objects, surfaces, and features (e.g., walls) in the interior of the body cavity and is reflected off objects, surfaces, and features (e.g., walls) in the interior of the body cavity. A portion of the reflected light may be collected through an aperture at the distal end of the elongate member 20. This light may be directed along an optical path through the elongate member 20 formed by the plurality of lenses disposed in the elongate tubular structure so as to form an image of the objects, surfaces, features at the proximal end of the endoscope 100. The light collected may then be directed to the imaging device (e.g., a detector such as a two-dimensional Charged Coupled Device (CCD) or Complementary Metal-Oxide Semiconductor (CMOS) detector array). Thus, an image of the object, surface, feature, etc. inside the body cavity can be viewed, for example, by the physician possibly on a display in communication (e.g., wireless communication) with the detector.

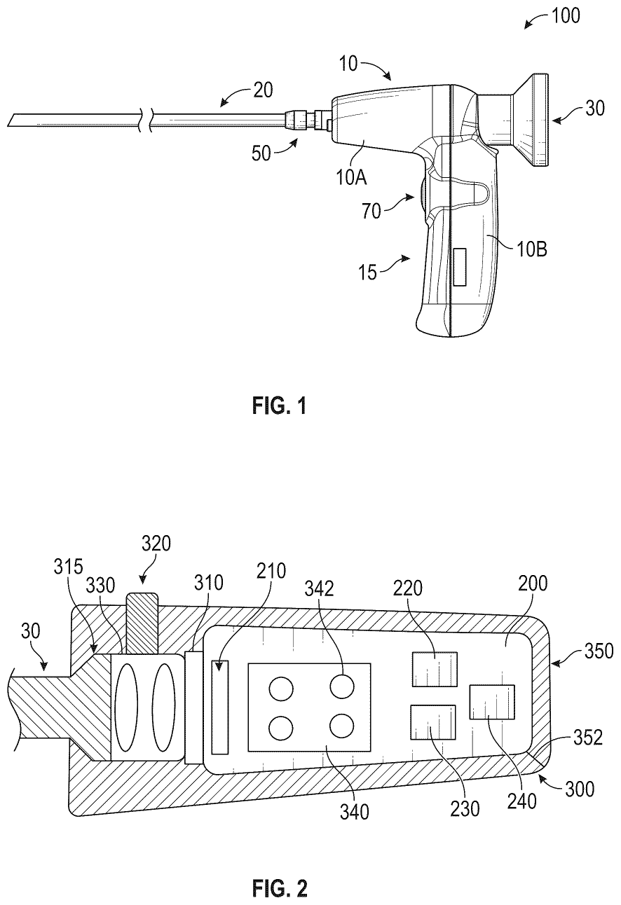

FIG. 2 shows a cover 300 that can fit (e.g. enclose) a camera 200 (e.g., a wireless camera). The cover 300 can removably receive or enclose the camera 200 and can be sterilized (e.g., provide a sterilized cover for the camera 200). The camera 200 can be or include an imaging device 210 (e.g., a detector such as a two-dimensional CCD or CMOS detector array) to sense the image relayed to the imaging device (e.g., by the endoscope to which the camera 200 is coupled). In the illustrated example, the cover 300 can removably couple to an eyecup 30 of an endoscope (e.g., the endoscope 100) via a connector 315. For example, the connector 315 of the cover 300 can have an opening sized to receive and retain (e.g., resiliently retain, such as via tapered side walls) the eyecup 30 to allow the cover 300 (and therefore camera 200) and endoscope to be an integral (e.g., connected, coupled) assembly. In one example, the cover 300 (housing the camera 200) and the endoscope can be operated (e.g., rotated) simultaneously (e.g., rotate together as a single unit). In another example, the connector 315 of the camera cover 300 couples to the eyecup 30 to retain the eyecup 30 coupled to the cover 300 while allowing the eyecup 30 (and therefore the endoscope tube 20 and endoscope 100) to be rotated relative to the cover 300 (e.g., manually rotated by the user, using the same hand as the one grasping the cover 300), for example to change a viewing angle of the endoscope to view a different portion of the surgical site. The connector 315 can have a window or opening/aperture that allows the image to pass from the endoscope to the image sensor when the endoscope is coupled to the connector 315.

The cover 300 can in one example be made of a resilient material (e.g., synthetic rubber). The cover 300 can optionally include (or accommodate therein) a connector 310 (e.g., a C-mount connector for coupling with a direct mount endoscope), a lens assembly 330 (e.g., lens coupler) with one or more lenses (e.g., with multiple lenses), an interface 320 (e.g., paddle, wheel, dial, lever) actuatable to adjust a focus of the camera 200 (e.g., by acting on, such as moving, sliding, rotating, the lens assembly 330), and a resilient (e.g., rubberized) window 340 (or panel) with buttons 342 to allow the actuation (e.g., pressing) of corresponding buttons on the camera 200 via the resilient window or panel 340 of the cover 300 (e.g., the buttons 342 align with corresponding buttons of the camera 200). In some examples, the buttons 342 can be made of a harder material (e.g., hard plastic) than the resilient window or panel 340 (e.g., which can be of rubber) to allow the buttons 342 to contact, bear on or otherwise actuate the buttons of the camera 200. In some embodiments, the cover 300 can exclude one or ore of the features discussed above (e.g., exclude the connector 310, the interface 320, the lens assembly 330 and/or the window 340). For example, in one example, the camera 200 can exclude an interface with buttons, and the camera cover 300 can exclude the window or panel 340 with buttons 342, and the camera 200 can be controlled wirelessly (e.g., via remote control), as further discussed below. In one example, the camera 200 can include batteries 220, circuitry 230 (e.g., printed circuit board (PCB)), and transceiver 240 for wireless communication (e.g., to communicate captured images wirelessly, such as to a video monitor, remote computer, etc.). The camera 200 can advantageously be removably inserted into the cover 300. For example, the cover 300 can have a panel (e.g., a flap, hatch) 350 that can be moved (pulled) relative to the rest of the cover 300 to allow access to the opening or cavity in the cover 300 to insert the camera 200 into the cover 300 or to remove the camera 200 from the cover 300. The panel (e.g., flap, hatch) 350 can resiliently close the opening and be biased toward a closed state where the panel (e.g., flap, hatch) 350 is in contact with the rest of the cover 300 (e.g., along an edge 352). The cover 300 is advantageously disposable and can be disposed after use (e.g., after a single use or procedure) after it is removed from over the camera 200, making it unnecessary to autoclave (or otherwise sterilize) the camera 200 between uses or surgical procedures.

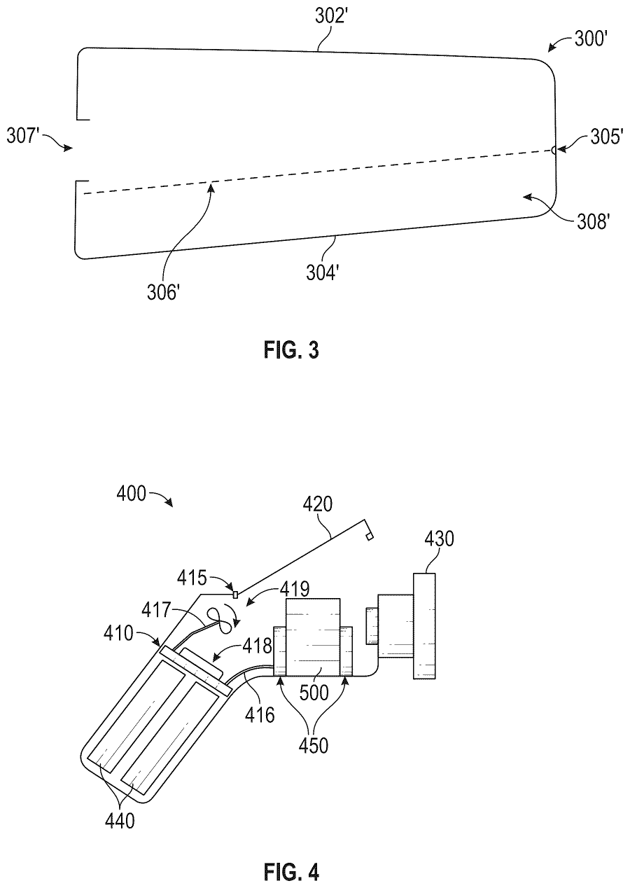

FIG. 3 shows a cover 300′ for a camera (e.g., wireless camera, such as camera 200). The cover 300′ can be a clamshell type cover with a first (e.g., upper) cover portion 302′ and a second (e.g., lower) cover portion 304′ connected at one end via a hinge 305′. In one example, the hinge 305′ is a living hinge. The first (e.g., upper) cover portion 302′ and the second (e.g., lower) cover portion 304′ can contact each other along an edge (or split line) 306′ when in the closed position. The first (e.g., upper) cover portion 302′ and the second (e.g., lower) cover portion 304′ can pivot away from each other via the hinge 305′ when in the open position to allow insertion of the camera into, or removal of the camera from, a space 308′ (e.g., cavity, chamber) defined by the first (e.g., upper) cover portion 302′ and the second (e.g., lower) cover portion 304′. The camera can at least partially reside in the space 308′ so that it extends into a space portion defined by the first (e.g., upper) cover portion 302′ and also extends into a space portion defined by the second (e.g., lower) cover portion 304′. The cover 300′ can be form fitting relative to the camera when in the closed position. The cover 300′ can define an opening 307′ at an end of the cover 300′ opposite the hinge 305′. In one implementation, the opening 307′ can be defined at least in part by the first (e.g., upper) cover portion 304′ and can be defined at least in part by the second (e.g., lower) cover portion 306′. The opening 307′ can be sized to couple to an endoscope. For example, the opening 307′ can extend over an eyecup of an endoscope (e.g., the eyecup 30 in FIG. 2) so that the eyecup is retained by the cover 300′ (e.g., retained in the space 308′) and so that the eyecup is inhibited (e.g., prevented) from inadvertently decoupling from the cover 300′. The cover 300′ is advantageously disposable and can be disposed after use (e.g., after a single use or procedure) after it is removed from over the camera (e.g., camera 200), making it unnecessary to autoclave (or otherwise sterilize) the camera between uses or surgical procedures. Though not shown, the cover 300′ can optionally have a connector, an interface (e.g., paddle, wheel, dial), a lens assembly (e.g., with one or more lenses) and/or a resilient window (e.g., like the connector 310, the interface 320, the lens assembly 330 and/or the window 340 in FIG. 2).

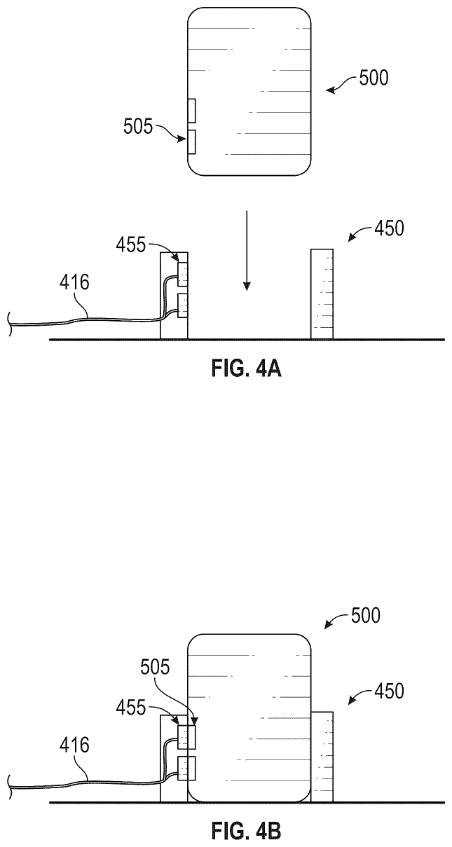

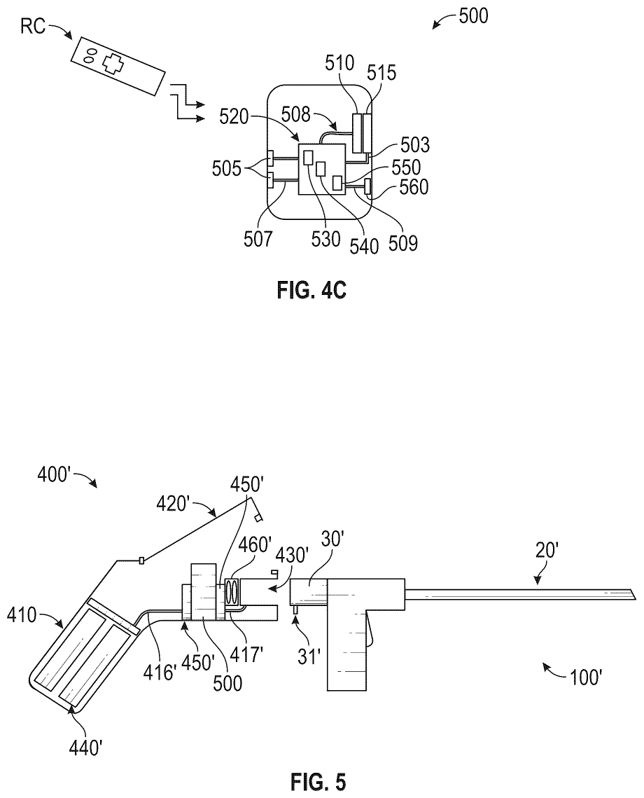

FIG. 4 shows a cover 400 with a housing 410 and a hatch 420 pivotally coupled to the housing 410 via a hinge 415 and movable between an open position (shown in FIG. 4) and a closed position. The housing 410 and the hatch 420 can be made of the same material, such as plastic (e.g., hard plastic). A connector 430 can be attached to the housing 410 for coupling with an endoscope. The connector 430 can be a reusable coupler (e.g., C-mount connector). The connector 430 can be a universal coupler for coupling to different endoscopes. The housing 410 can house electronics 418 (e.g., printed circuit board (PCB) with circuitry thereon) and one or more (e.g., two) batteries 440 (e.g., lithium ion, alkaline, rechargeable). The connector 430 can have a window or opening/aperture that allows the image to pass from the endoscope to the image sensor, when the endoscope is coupled to the connector 430. The housing 410 can removably receive a non-sterile camera module 500 (e.g., when the hatch 420 is in the open position) and enclose the camera module 500 (e.g., when the hatch 420 is in the closed position). The cover 400 can have indexing features 450 for indexing the camera module 500. The indexing feature 450 can be a slot that receives the camera module 500. In one example, the cover 400 can include a fan 419 electrically connected (via wiring 417) to the electronics 418 and batteries 440 and operable to dissipate heat from within the cover 400. In another example, the fan 419 and wiring 417 are excluded. In one example, the batteries 440 and/or electronics 418 can be electrically connected to the indexing feature (e.g., slot) 450 via wiring 416. As shown in FIGS. 4A-4B, the camera module 500 can be powered by the batteries 440 via electrical contacts 455 in the indexing feature (e.g., slot) 450 electrically connected to the batteries 440 via the wiring 416. The electrical contacts 455 can contact electrical contacts 505 in the camera module 500 once the camera module 500 is seated in the indexing feature (e.g. slot) 450 to thereby provide power from the batteries 440 to the camera module 500. The electronics 418 can in one example control power delivery from the batteries, and/or provide wireless communication, etc. and the camera module 500 can exclude circuitry or a transceiver/transmitter for wireless communication.

With reference to FIG. 4C, in one example the camera module 500 can include imaging device 510 (e.g., a detector or sensor, such as a two-dimensional CCD or CMOS detector array), electronics 520 (e.g., printed circuit board with circuitry), for example including a transmitter or a transceiver 530 for wireless communication, a processor 540 and a memory 550. In one example, the cover 400 can exclude the electronics 418, and the electronics in the camera module 500 control power delivery from the batteries 440 and wireless communication. The electronics 520 are electrically connected to the electrical contacts 505 by wiring 507 and the imaging device 510 is electrically connected to the electronics 520 by wiring 508. Optionally, the camera module 500 can have a built-in lens coupler 515 (e.g. electronically controlled focus) that is electrically connected to the electronics 520 by wiring 503. The electrical contacts 505 facilitate control of electrical components in the endoscope or electronic lens coupler, as further discussed below. In one example, the electronics 520 are connected to an electrical contact 560 by wiring 509 to electrically connect the camera module 500 with an endoscope, as further discussed below. In one example, the camera module 500 can be controlled by a remote control RC (e.g., via the transceiver of the camera module 500 so that the cover 400 and camera module 500 can exclude buttons, such as the buttons 342 in FIG. 2 for manual actuation of the camera). For example, the remote control RC can be operable to actuate the camera module 500 to take still images, to turn camera on or off, to being or top a video recording, etc. Where the camera module 500 is not controlled by remote control RC, in one example the camera module 500 can have a transmitter instead of a transceiver. The imaging device 510 can communicate captured images (wirelessly) via the electronics 520 (e.g., transceiver or transmitter).

The cover 400 (e.g., housing 410, hatch 420, batteries 440) is advantageously disposable and can be disposed after use (e.g., after a single use or procedure) after the camera module 500 is removed from the cover 400 (e.g., and the connector 430 is removed), making it unnecessary to autoclave (or otherwise sterilize) the camera module 500 between uses or surgical procedures. Rather, the non-sterile camera module 500 can be reused by installing it within another cover 400 prior to a subsequent use or surgical procedure. In this manner, the camera module 500 is not subjected to the harsh autoclaving (or other sterilization) procedure, thereby reducing wear and tear and maintenance needed on the camera module 500, prolonging the life of the camera module 500. Additionally, the same camera module 500 can be used with multiple covers 400, thereby reducing operating costs.

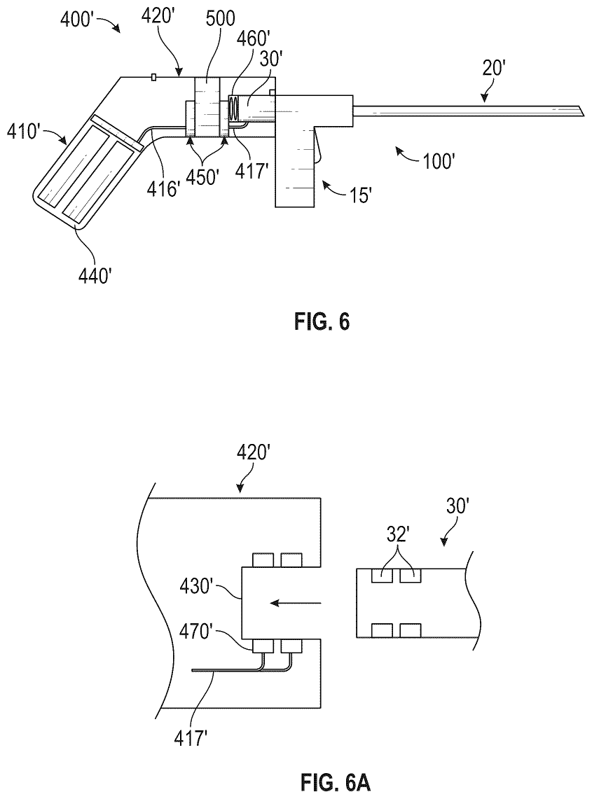

FIGS. 5-6 show a cover 400′ for a camera module 500. Some of the features of the cover 400′ are similar to features of the cover 400 in FIG. 4. Thus, reference numerals used to designate the various components of the cover 400′ are identical to those used for identifying the corresponding components of the cover 400 in FIG. 4, except that a “′” has been added to the numerical identifier. Therefore, the structure and description for the various features of the cover 400 and how it's operated and controlled in FIG. 4 are understood to also apply to the corresponding features of the cover 400′ in FIG. 5, except as described below.

The cover 400′ differs from the cover 400 in that it has a connector 430′ that directly connects to a connector 30′ of an endoscope 100′. Additionally, the cover 400′ excludes a fan or electronics (e.g., PCB, circuitry) in the cover 400′. The batteri(es) 440′ are electrically connected by wiring 416′ to the camera module 500 in the manner described above for FIGS. 4A-4C. The connector 430′ can be a recessed (e.g., female) connector that receives the connector 30′ of the endoscope 100′. The connector 430′ can have a window or opening/aperture that allows the image to pass from the endoscope to the image sensor (e.g., of the camera module 500). In one example, the connector 430′ and the connector 30′ can define a threaded coupling. In another example, the connector 30′ of the endoscope 100′ is a bayonet connector with one or more keys 31′ that fit in slots (not shown) of the connector 430′. In one example, best shown in FIG. 6A, the endoscope 100′ can be powered by the batteri(es) 440′ via wiring 417′ that extends between and electrically connects the electrical contact(s) 505 of the camera module 500 (see FIG. 4C) with electrical contacts 470′ (e.g., ring contacts) of the connector 430′. The electrical contacts 470′ of the connector 430′ can electrically contact corresponding contacts 32′ (e.g., ring contacts) in or on the connector 30′ of the endoscope 100′. Advantageously, such an arrangement allows for the simplification of the endoscope 100′, as electronics (e.g., PCB, circuitry) and batteri(es) can be excluded from the endoscope 100′ and the light source(s) (e.g., LEDs) of the endoscope 100′ can be powered by the batteri(es) 440′ in the cover 400′ and controlled by the electronics 520 of the camera module 500 via the wiring 417′ and electrical connection between the contacts 470′ of the connector 430′ of the cover and the connector 30′ of the endoscope 100′. In another example, the endoscope 100′ includes electronics and batteri(es), as discussed above in connection with the endoscope 100, and is not powered by the batteri(es) 440 or controlled by electronics in the camera module 500 or cover 400′.

Additionally, the cover 400′ can have either a built in or removable (e.g., disposable) coupler 460′ with one or more lenses (e.g., focusing is not done in the endoscope or with a coupler to couples between the camera cover and the endoscope). The coupler 460′ can align with the camera module 500 (e.g., align with the image sensor or imaging device 510 of the camera module 500) when the camera module 500 is inserted into the cover 400′ (e.g., when the camera module 500 is seated in the indexing feature or slot 450′). The coupler 460′ can be actuated mechanically (e.g., by a lever, paddle, wheel, dial, such as interface 320 above) to adjust focus. In another example, the coupler 460′ can be electronically controlled to adjust focus. For example, the coupler 460′ can have electrical contacts that electrically connect to the contact(s) 505 of the camera module 500, thereby allowing the camera module 500 (e.g., the electronics of the camera module 500) to electronically control focus adjustment of the coupler 460′. In another example, the camera module 500 has a built-in lens coupler and the cover 400′ excludes the coupler 460′,

The cover 400′ (e.g., housing 410′, hatch 420′, batteries 440′) is advantageously disposable and can be disposed after use (e.g., after a single use or procedure) after the camera module 500 is removed from the cover 400′, making it unnecessary to autoclave (or otherwise sterilize) the camera module 500 between uses or surgical procedures. Rather, the non-sterile camera module 500 can be reused by installing it within another cover 400′ prior to a subsequent use or surgical procedure. Optionally, the batteries 440′ are not disposed after a single use and can be removed from the cover 400′, recharged and reinserted into a new cover 400. In this manner, the camera module 500 is not subjected to the harsh autoclaving (or other sterilization) procedure. Additionally, the same camera module 500 can be used with multiple covers 400′, thereby reducing operating costs. Optionally, the same camera module 500 can be used with the cover 400 and with the cover 400′, thereby accommodating use of the camera module 500 with different endoscope designs.

With continued reference to FIG. 6, the cover 400′ with the recessed connector 430′ advantageously allows the endoscope 100′ to be rotated while coupled to the cover 400′, since it allows the connector 30′ to extend into the cover 400′ and for the handle 15′ of the endoscope 100′ to be closer or proximate (e.g., adjacent, next to) an end of the cover 400′. For example, the user (e.g. surgeon) can rotate the endoscope 100′ about its axis to adjust the viewing angle of the endoscope 100′ (e.g., to view a different portion of the surgical site in which the endoscope is inserted) since the end of the endoscope tube 20′ is angled or beveled. The user (e.g., surgeon) can manually rotate the endoscope 100′ with the same hand used to hold the cover 400′ containing the camera module 500. For example, while holding the cover 400′, the user (e.g., surgeon) can rotate the endoscope 100 by extending their index and/or middle finger to contact and bear against the side of the handle 15′, and pushing on the handle 15′ with such finger(s) to rotate the handle 15′, and therefore the endoscope tube 20′, about the axis of the endoscope tube 20′.

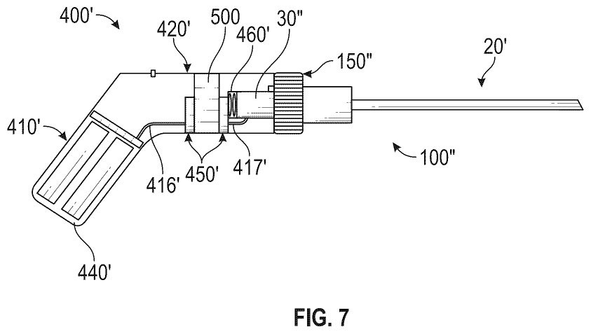

FIG. 7 shows the cover 400′, as described above, coupled to an endoscope 100″. Some of the features of the endoscope 100″ are similar to features of the endoscope 100′ in FIGS. 5-6. Thus, reference numerals used to designate the various components of the endoscope 100″ are identical to those used for identifying the corresponding components of the endoscope 100′ in FIGS. 5-6, except that a “′” has been added to the numerical identifier. Therefore, the structure and description for the various features of the endoscope 100′ and how it's operated and controlled in FIGS. 5-6 are understood to also apply to the corresponding features of the endoscope 100″ in FIG. 7, except as described below.

The endoscope 100″ differs from the endoscope 100′ in that it excludes a handle (e.g., handle 15′). The endoscope 100″ connects via the connector 30″ to the connector 430′ of the cover 400′. In one example, the endoscope 100″ excludes batteri(es) or electronics (e.g., PCB, circuitry) and is instead powered by the batteri(es) 440′ and controlled by electronics 520 in the camera module 500 or cover 400′. In another example, the endoscope 100″ includes electronics and batteri(es), as discussed above in connection with the endoscope 100, and is not powered by the batteri(es) 440 or controlled by electronics in the camera module 500 and/or cover 400′.

The cover 400′ with the recessed connector 430′ advantageously allows the endoscope 100″ to be rotated while coupled to the cover 400′, since it allows the connector 30″ to extend into the cover 400′ and a ring 150″ (e.g., knurled ring) of the endoscope 100″ to be closer or proximate (e.g., adjacent, next to) an end of the cover 400′. For example, the user (e.g. surgeon) can rotate the endoscope 100″ about its axis to adjust the viewing angle of the endoscope 100″ (e.g., to view a different portion of the surgical site in which the endoscope is inserted) since the end of the endoscope tube 20″ is angled or beveled. The user (e.g., surgeon) can manually rotate the endoscope 100″ with the same hand used to hold the cover 400′ containing the camera module 500. For example, while holding the cover 400′, the user (e.g., surgeon) can rotate the endoscope 100 by extending their index and/or middle finger to contact and bear against a surface of the ring 150″ to rotate the ring 150″ with such finger(s) to rotate the endoscope tube 20′ about the axis of the endoscope tube 20′ to change the viewing angle of the endoscope 100″. With reference to FIGS. 5-7, the direct mount connection of the endoscope 100′, 100″ with the cover 400′ advantageously, inhibits (e.g., prevents) fogging of the lenses in the lens coupler. In one example, with reference to FIGS. 6-7, the endoscope 100′, 100″ can be permanently coupled to the cover 400′, and the cover 400′ can be disposed with the endoscope 100′, 100″ after use (e.g., after a single use), such as after the camera module 500 is removed (and optionally the batteri(es) 400′ removed).

Advantageously, the cover 300, 300′, 400, 400′ can removably receive a camera (e.g., camera 200, camera module 500), and can be disposed after use (e.g., after a single use or procedure) after the camera is removed from the cover 300, 300′, 400, 400′, making it unnecessary to autoclave (or otherwise sterilize) the camera between uses or surgical procedures. Rather, the non-sterile camera can be reused by installing it within another cover 300, 300′, 400, 400′ prior to a subsequent use or surgical procedure. The cover can advantageously accommodate different endoscope designs (e.g., couple to an eyepiece of an endoscope, couple to a direct mount endoscope) and lens coupler designs (e.g. have an integrated lens coupler, have a removable lens coupler, have a detachable lens coupler that is disposed between the cover containing the camera and the endoscope). Advantageously, the cover 300, 300′, 400, 400′ can be made of a material with good (e.g., high thermal conductivity), such as metal (e.g., aluminum) or a polymer material. Advantageously, the cover 300, 300′, 400, 400′ is waterproof and inhibits (e.g. prevents) water or other liquid from entering the cover 300, 300′, 400, 400′ (e.g., when the cover is in the closed position).

### Additional Embodiments

In embodiments of the present disclosure, a disposable cover for a wireless camera, a wireless camera assembly (with or without endoscope) may be in accordance with any of the following clauses:
Clause 1. A wireless camera assembly, comprising: a disposable cover including a housing and a hatch movable relative to the housing between an open position allowing access to a cavity in the housing and closed position to close the housing, the housing including a connector configured to couple to a proximal end of an endoscope, the housing having a window or opening via which an image passes into the housing from the endoscope, and the disposable cover configured to receive a wireless camera in the cavity of the disposable cover when the hatch is in the open position, the cover configured to align an imaging device of the wireless camera with the window of the housing and with the endoscope when the endoscope is coupled to the connector and when the wireless camera is inserted into the cavity.
Clause 2. The assembly of Clause 1, wherein the disposable cover has a clamshell form with a first housing portion pivotally coupled to a second housing portion, the cover being disposable after a single use.
Clause 3. The assembly of any preceding clause, wherein the housing includes one or more batteries in the cavity, and wherein the wireless camera is a wireless camera module that includes the imaging device and a transceiver, the wireless camera module configured to receive power from the one or more batteries in the housing.
Clause 4. The assembly of Clause 3, wherein the wireless camera module includes a lens assembly operable to focus an image on the imaging device.
Clause 5. The assembly of Clause 3, wherein the wireless camera module includes circuitry operable to control one or more LEDs of the endoscope when the endoscope is coupled to the connector of the housing, the one or more batteries configured to power the LEDs of the endoscope via an electrical connection between electrical contacts of the connector of the housing and corresponding electrical connectors of a connector at the proximal end of the endoscope.
Clause 6. The assembly of Clause 3, wherein the housing has an indexing slot configured to removably receive the wireless camera module.
Clause 7. The assembly of Clause 3, wherein wireless camera module electrically connects with the one or more batteries via one or more electrical contacts in the housing and one or more corresponding electrical contacts of the wireless camera module when the wireless camera module is inserted into the housing, and wherein the wireless camera module electrically disconnects from the one or more batteries when the wireless camera module is removed from the housing.
Clause 8. The assembly of any preceding clause, wherein the connector of the cover is a direct mount connector that connects to a connector of the endoscope.
Clause 9. The assembly of any of Clauses 1-7, wherein the connector of the cover includes tapered side walls configured to engage an eyepiece of the endoscope.
Clause 10. The assembly of any preceding clause, wherein the disposable cover is form-fitting relative to the camera and comprises a resilient and heat conductive material.
Clause 11. The assembly of any preceding clause, wherein the disposable cover includes a lens assembly configured to align with the camera when the camera is inserted into the cover and configured to align with the endoscope when the endoscope is coupled to the connector.
Clause 12. The assembly of any preceding clause, wherein the connector of the housing is a recessed connector configured to receive and couple to a male connector of the endoscope.
Clause 13. The assembly of Clause 12, wherein the endoscope is manually rotatable by a user about an axis of an endoscope tube of the endoscope with a same hand holding the cover when the endoscope is coupled to the connector of the housing to vary a view angle of the endoscope.
Clause 14. A wireless camera assembly, comprising: a disposable cover including a housing and a hatch movable relative to the housing between an open position allowing access to a cavity in the housing and closed position to close the housing, the housing including a connector; an endoscope having a proximal end coupled to the connector of the housing, the endoscope being manually rotatable by a user about an axis of an endoscope tube of the endoscope with a same hand holding the cover to vary a view angle of the endoscope; and a wireless camera removably insertable in the cavity of the disposable cover, the camera insertable into the housing or removable from the housing when the hatch is in the open position, an imaging device of the wireless camera configured to align with the endoscope when the wireless camera is inserted into the cavity, the imaging device configured to receive an image from the endoscope.
Clause 15. The assembly of Clause 14, wherein the disposable cover and endoscope are disposable after a single use.
Clause 16. The assembly of any of Clauses 14-15, wherein the housing houses one or more batteries, the wireless camera being a wireless camera module that includes the imaging device and a transceiver, the wireless camera module insertable in an indexing slot of the housing and configured to receive power from the one or more batteries in the housing via electrical contacts of the indexing slot.
Clause 17. The assembly of Clause 16, wherein the wireless camera module includes a lens assembly operable to focus an image on the imaging device.
Clause 18. The assembly of Clause 16, wherein the wireless camera module includes circuitry operable to control one or more LEDs of the endoscope when the endoscope is coupled to the connector of the housing, the one or more batteries configured to power the LEDs of the endoscope via an electrical connection between electrical contacts of the connector of the housing and corresponding electrical connectors of a connector at the proximal end of the endoscope.
Clause 19. The assembly of Clause 16, wherein the wireless camera module electrically connects with the one or more batteries via one or more electrical contacts of the housing and of the wireless camera module when the wireless camera module is inserted into the housing and electrically disconnects from the one or more batteries when the wireless camera module is removed from the housing.
Clause 20. The assembly of Clause 16, wherein the endoscope is removably coupled to the housing and configured to be decoupled from the housing.

While certain embodiments of the inventions have been described, these embodiments have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

Of course, the foregoing description is that of certain features, aspects and advantages of the present invention, to which various changes and modifications can be made without departing from the spirit and scope of the present invention. Moreover, the devices described herein need not feature all of the objects, advantages, features and aspects discussed above. Thus, for example, those of skill in the art will recognize that the invention can be embodied or carried out in a manner that achieves or optimizes one advantage or a group of advantages as taught herein without necessarily achieving other objects or advantages as may be taught or suggested herein. In addition, while a number of variations of the invention have been shown and described in detail, other modifications and methods of use, which are within the scope of this invention, will be readily apparent to those of skill in the art based upon this disclosure. It is contemplated that various combinations or subcombinations of these specific features and aspects of embodiments may be made and still fall within the scope of the invention. Accordingly, it should be understood that various features and aspects of the disclosed embodiments can be combined with or substituted for one another in order to form varying modes of the discussed devices.

What is claimed is:

1. A wireless camera assembly, comprising:
a disposable cover including a housing and a hatch movable relative to the housing between an open position allowing access to a cavity in the housing and closed position to close the housing, the housing including a connector configured to couple to a proximal end of an endoscope, the housing having a window or opening via which an image passes into the housing from the endoscope, and
the disposable cover configured to receive a wireless camera in the cavity of the disposable cover when the hatch is in the open position, the cover configured to align an imaging device of the wireless camera with the window of the housing and with the endoscope when the endoscope is coupled to the connector and when the wireless camera is inserted into the cavity.

2. The assembly of claim 1, wherein the disposable cover has a clamshell form with a first housing portion pivotally coupled to a second housing portion, the cover being disposable after a single use.

3. The assembly of claim 1, wherein the housing includes one or more batteries in the cavity, and wherein the wireless camera is a wireless camera module that includes the imaging device and a transceiver, the wireless camera module configured to receive power from the one or more batteries in the housing.

4. The assembly of claim 3, wherein the wireless camera module includes a lens assembly operable to focus an image on the imaging device.

5. The assembly of claim 3, wherein the wireless camera module includes circuitry operable to control one or more LEDs of the endoscope when the endoscope is coupled to the connector of the housing, the one or more batteries configured to power the LEDs of the endoscope via an electrical connection between electrical contacts of the connector of the housing and corresponding electrical connectors of a connector at the proximal end of the endoscope.

6. The assembly of claim 3, wherein the housing has an indexing slot configured to removably receive the wireless camera module.

7. The assembly of claim 3, wherein wireless camera module electrically connects with the one or more batteries via one or more electrical contacts in the housing and one or more corresponding electrical contacts of the wireless camera module when the wireless camera module is inserted into the housing, and wherein the wireless camera module electrically disconnects from the one or more batteries when the wireless camera module is removed from the housing.

8. The assembly of claim 1, wherein the connector of the cover is a direct mount connector that connects to a connector of the endoscope.

9. The assembly of claim 1, wherein the connector of the cover includes tapered side walls configured to engage an eyepiece of the endoscope.

10. The assembly of claim 1, wherein the disposable cover is form-fitting relative to the camera and comprises a resilient and heat conductive material.

11. The assembly of claim 1, wherein the disposable cover includes a lens assembly configured to align with the camera when the camera is inserted into the cover and configured to align with the endoscope when the endoscope is coupled to the connector.

12. The assembly of claim 1, wherein the connector of the housing is a recessed connector configured to receive and couple to a male connector of the endoscope.

13. The assembly of claim 12, wherein the endoscope is manually rotatable by a user about an axis of an endoscope tube of the endoscope with a same hand holding the cover when the endoscope is coupled to the connector of the housing to vary a view angle of the endoscope.

14. A wireless camera assembly, comprising:
a disposable cover including a housing and a hatch movable relative to the housing between an open position allowing access to a cavity in the housing and closed position to close the housing, the housing including a connector;
an endoscope having a proximal end coupled to the connector of the housing, the endoscope being manually rotatable by a user about an axis of an endoscope tube of the endoscope with a same hand holding the cover to vary a view angle of the endoscope; and
a wireless camera removably insertable in the cavity of the disposable cover, the camera insertable into the housing or removable from the housing when the hatch is in the open position, an imaging device of the wireless camera configured to align with the endoscope when the wireless camera is inserted into the cavity, the imaging device configured to receive an image from the endoscope.

15. The assembly of claim 14, wherein the disposable cover and endoscope are disposable after a single use.

16. The assembly of claim 14, wherein the housing houses one or more batteries, the wireless camera being a wireless camera module that includes the imaging device and a transceiver, the wireless camera module insertable in an indexing slot of the housing and configured to receive power from the one or more batteries in the housing via electrical contacts of the indexing slot.

17. The assembly of claim 16, wherein the wireless camera module includes a lens assembly operable to focus an image on the imaging device.

18. The assembly of claim 16, wherein the wireless camera module includes circuitry operable to control one or more LEDs of the endoscope when the endoscope is coupled to the connector of the housing, the one or more batteries configured to power the LEDs of the endoscope via an electrical connection between electrical contacts of the connector of the housing and corresponding electrical connectors of a connector at the proximal end of the endoscope.

19. The assembly of claim 16, wherein the wireless camera module electrically connects with the one or more batteries via one or more electrical contacts of the housing and of the wireless camera module when the wireless camera module is inserted into the housing and electrically disconnects from the one or more batteries when the wireless camera module is removed from the housing.

20. The assembly of claim 16, wherein the endoscope is removably coupled to the housing and configured to be decoupled from the housing.
